(19) United States
(12) Patent Application Publication
(10) Pub. No.: US 2009/0118711 A1
(43) Pub. Date: May 7, 2009

(54) REDUCED-NOISE IMPLANTABLE INFUSION DEVICE

(75) Inventors: James M. Haase, Maplewood, MN (US); Ronald L. Mezera, Lake Elmo, MN (US); Frederick John Gray, Woodland Hills, CA (US); Zbynek Struzka, Fribourg (CH)

Correspondence Address: CAMPBELL NELSON WHIPPS, LLC, 408 ST. PETER STREET, SUITE 240, ST. PAUL, MN, 55102, US

(73) Assignee: MEDTRONIC, INC., Minneapolis, MN (US)

(21) Appl. No.: 12/334,985

(22) Filed: Dec. 15, 2008

Related U.S. Application Data
(63) Continuation-in-part of application No. 11/171,587, filed on Jun. 29, 2005.
Application No. 11/171,587 is a division of application No. 10/033,722, filed on Dec. 27, 2001, now Pat. No. 6,997,921.
(60) Provisional application No. 60/317,886, filed on Sep. 7, 2001.

(51) Int. Cl. A61K 9/22

(52) U.S. Cl. 604/891.1

(57) ABSTRACT

A drive mechanism for delivery of infusion medium in an implantable medical device includes an inlet for receiving the infusion medium and a piston channel for communication of infusion medium received by the inlet. The piston channel has a distal end and a proximal end. The proximal end is closer to the inlet than the distal end. The drive mechanism further includes a coil surrounding the piston channel and a piston located within the piston channel and moveable axially within the piston channel to drive infusion medium out of the distal end of the piston channel. The mechanism also includes an armature operably coupled to the piston and disposed adjacent the coil. The armature has first and second opposing major surfaces and a plurality of vents extending through the armature from the first major surface to the second major surface. The plurality of vents cumulatively occupy between about 20% and about 40% of the total surface area of the first major surface. Electromagnetic interaction between the armature and the coil cause the piston move in the channel.

### RELATED APPLICATION

This application is a Continuation-In-Part of application Ser. No. 11/171,587, filed Jun. 29, 2005, which is a divisional of application Ser. No. 10/033,722, flied Dec. 27, 2001, now U.S. Pat. No. 6,997,921, which claims priority to provisional U.S. Application Ser. No. 60/317,886, filed Sep. 7, 2001. The entire contents of each are hereby incorporated herein by reference to the extent that they do not conflict with the disclosure presented herein.

### FIELD

The present disclosure relates generally to infusion devices, systems and processes and, in particular embodiments to implantable infusion devices, systems and processes employing a piston drive mechanism configuration which allows the device to use power efficiently and to operate quietly.

### BACKGROUND

Infusion devices are typically used to deliver an infusion media, such as a medication, to a patient. Implantable infusion devices are designed to be implanted in a patient's body and to administer an infusion media to the patient at a regulated dosage.

In some contexts of use, the implantable infusion device is configured to be operable for an extended period with a limited power supply. For example, battery powered infusion devices may be implanted in patients, to deliver medication at controlled intervals over a prolonged period of time. As the battery power supplies for such devices have limited capacities, some devices typically require multiple replacements of batteries over their operational life. Others require periodic recharging. Accordingly, it is desirable for implantable infusion devices to operate efficiently and, thus, require fewer power supply replacements or recharges.

Some implantable infusion devices employ a piston drive mechanism. Such devices can produce an audible noise upon activation as the moving actuator strikes a stationary pump structure. The noise may not be well tolerated by the patient and may impact the patient's quality of life. Reducing such noise while maintaining energy efficiency presents a difficult challenge.

### BRIEF SUMMARY

The present disclosure describes piston drive mechanisms for infusion devices that allow the infusion device to operate in an efficient manner and to operate quietly.

In various embodiments, drive mechanisms include an inlet for receiving the infusion medium and a piston channel for communication of infusion medium received by the inlet. The piston channel has a distal end and a proximal end. The proximal end is closer to the inlet than the distal end. The drive mechanism further includes a coil surrounding the piston channel and a piston located within the piston channel and moveable axially within the piston channel to drive infusion medium out of the distal end of the piston channel. The mechanism also includes an armature operably coupled to the piston and disposed adjacent the coil. The armature has first and second opposing major surfaces and a plurality of vents extending through the armature from the first major surface to the second major surface. The plurality of vents cumulatively occupy between about 20% and about 40% of the total surface area of the first major surface. Electromagnetic interaction between the armature and the coil cause the piston move in the channel.

Drive mechanisms having armatures as described herein may operate in an energy efficient and quiet manner. These and other aspects and advantages will be apparent to one of skill in the art from the accompanying detailed description and drawings.

### BRIEF DESCRIPTION OF THE DRAWINGS

Referring now to the drawings in which like reference numbers represent corresponding parts throughout:

FIG. 1 is a block diagram illustrating some components of a representative infusion device.

FIG. 2 is a block diagram illustrating some components of a representative drive mechanism.

FIGS. 3A-B are schematic diagrams of cross sections illustrating some representative components of a drive mechanism.

FIG. 4 is a schematic perspective view of a representative actuator having a piston portion and an armature portion.

FIGS. 5-7 are schematic head-on top-down (according to orientation in FIG. 4) views of representative armature portions actuators.

FIG. 8 is a schematic perspective view of a representative actuator having a piston portion and an armature portion.

FIGS. 9-11 are schematic head-on top-down (according to orientation in FIG. 8) views of representative armature portions actuators.

FIG. 12 is a schematic cross-section view of one example embodiment of the drive mechanism in a retracted position or state.

FIG. 13 is a schematic cross-section view of the example drive mechanism embodiment of FIG. 12, in a forward stroke position or state.

FIG. 14 is a schematic of an exploded view of an embodiment of the drive mechanism shown in FIGS. 12 and 13.

FIG. 15 is a schematic perspective view of an embodiment of the inlet end of a housing for the drive mechanism in FIGS. 12 and 13.

FIG. 16 is a schematic perspective view of an embodiment of the outlet end of the drive mechanism housing of FIG. 15.

FIG. 17 is a schematic perspective view of an embodiment of a coil cup for the drive mechanism in FIGS. 12 and 13.

The drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope of spirit of the present invention. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

As used herein, “about” means +/−10% of the enumerated numerical value that it preceeds.

As discussed above, the present disclosure relates generally to infusion devices having drive mechanisms and also to drive mechanism configurations for infusion of a medium into a patient or other environment. In various embodiments, such devices and drive mechanisms are configured for implantation in a patient's body. The devices and drive mechanisms are configured to operate in a power efficient manner and to operate quietly.

As described with regard to various embodiments herein, the drive mechanisms may be piston-type drive mechanisms having an actuator that includes an armature coupled to a piston. The actuator moves from a retracted position to a forward position, driving fluid through a stationary channel. The piston is axially moveable within the channel. As the actuator moves from the retracted position to the forward position, the armature may contact a stationary member of the drive mechanism that forms the channel, causing an audible noise. With such piston-type drive mechanisms, counteracting noise often results in increased energy consumption. For example, as the armature approaches the stationary member, the armature would be expected to increase speed due to increased electromagnetic force, absent fluidic resistance between the armature and the stationary member. Thus, by increasing fluidic resistance between the armature and the stationary member, the armature may be slowed as it approaches the stationary part of the drive mechanism, thereby reducing noise from the interaction of the drive mechanism and the stationary member. However, increased energy consumption may be required to overcome the increased fluidic resistance, resulting in an inefficient device. As described in more detail below, it has been found that armatures having a plurality of vents that cumulatively occupy between about 20% and about 40% of the total surface area of a major surface of the armature can result in a quiet and energy efficient drive mechanism.

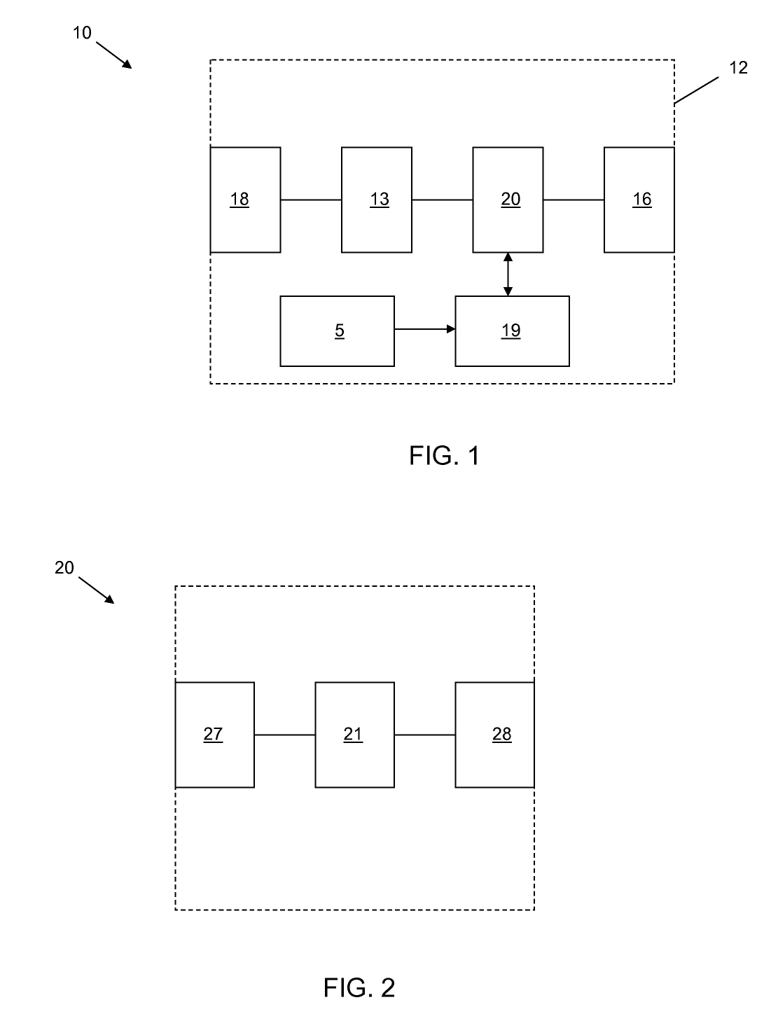

Prior to describing details regarding such armatures, a general discussion of representative infusion devices and drive mechanisms is provided. Referring to FIG. 1, a block diagram of some components of a representative infusion device 10 is shown. The device includes a housing 12. Housing may be made of any suitable material, such as a rigid polymeric material or metallic material, such as titanium. If device 10 is configured to be implanted in a patient, the housing 12 is preferably hermetically sealed. In the depicted device 10, a fluid flow path includes an inlet 18, a reservoir 13, a drive mechanism 20, and an outlet 16. The inlet 18 is fluidly coupled with reservoir 13, in which infusion medium may be stored. The inlet 18 may include a port through the housing 12 to allow access to the reservoir 18 or fluid path upstream of the reservoir. A septum may be disposed over the port to seal the inlet 18. The reservoir 13 is fluidly coupled to the drive mechanism 20, which may draw infusion medium from the reservoir 13. The drive mechanism 20 is fluidly coupled to the outlet 16 and forces fluid drawn from the reservoir 12 out of the device 12 via the outlet 16. The outlet may include a port to which a catheter may be operably coupled. The infusion device 10 may include other components, such as valves, that are not shown in FIG. 1. For example, a one-way valve may be disposed between the inlet 18 and the reservoir 13 or between the drive mechanism 20 and the outlet 16.

Representative examples of reservoirs which may be employed in embodiments of infusion devices are described in U.S. Published Patent Application 2003/0050623, published Mar. 13, 2003, and entitled “Infusion Device And Reservoir For Same,” which is incorporated herein by reference in its entirety to the extent that it does not conflict with the present disclosure. However, further embodiments may employ other suitable reservoir configurations, including, but not limited to, those described in U.S. Pat. No. 5,514,103 and U.S. Pat. No. 5,176,644, each to Srisathapat et al, U.S. Pat. No. 5,167,633 to Mann et al., U.S. Pat. No. 4,697,622 to Swift and U.S. Pat. No. 4,573,994 to Fischell et al.

Examples of inlet structures are described in U.S. Pat. No. 7,186,236 to Gibson et al., entitled “Infusion Device And Inlet For Same;” U.S. Pat. No. 5,514,103 and U.S. Pat. No. 5,176,644, each to Srisathapat et al. U.S. Pat. No. 5,167,633 to Mann et al.; U.S. Pat. No. 4,697,622 to Swift; and U.S. Pat. No. 4,573,994 to Fischell et al.

Still referring to FIG. 1, the infusion device 10 includes electronics 19 and a power source 5 disposed in the housing 12. The electronics 19 are operably coupled to the power source 18 and are configured to control the drive mechanism 20. The power source 18 may include a battery, such as a rechargeable battery. Electronics 18 may include a processor for controlling the drive mechanism 20; memory for storing instructions, recording diagnostics, or the like; a telemetry module for wireless communication; a diagnostics module; or the like. Such modules and electronic components are well known to those of skill in the art and may be readily included and employed as desired.

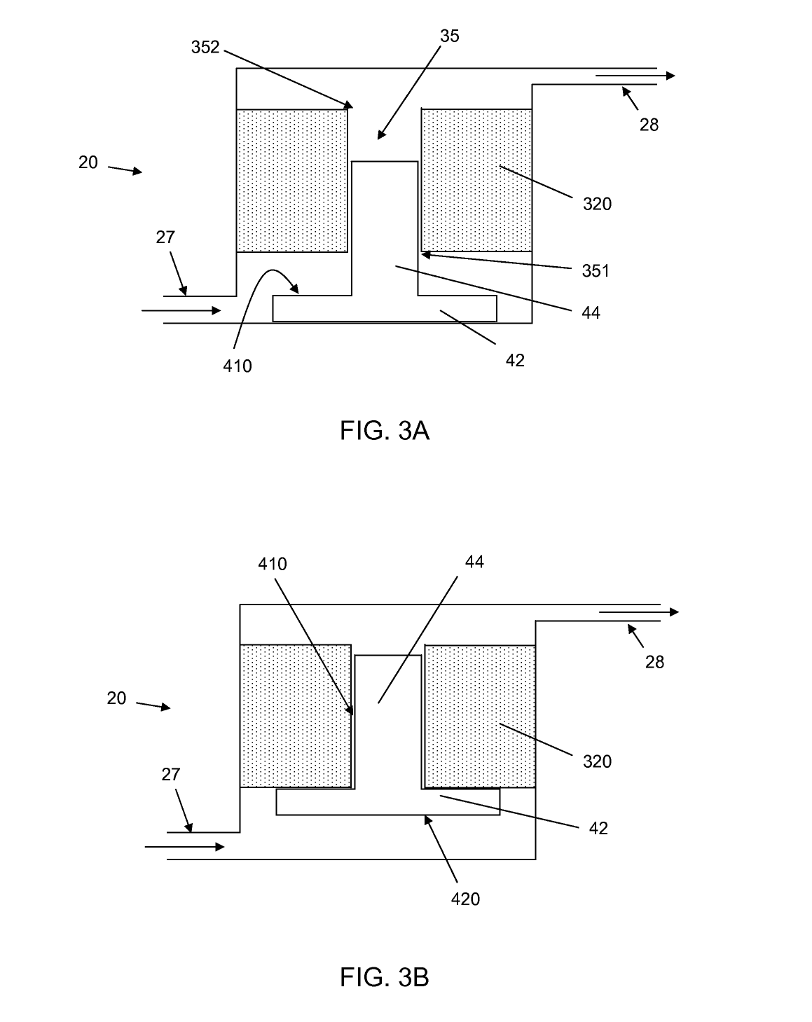

Referring now to FIGS. 2-3, representative components of piston drive mechanisms 20 are shown. In the block diagram of FIG. 2, the drive mechanism 20 includes an inlet 27, an outlet 28, and piston actuation mechanism 21. The piston actuation mechanism 21 is disposed in the flow path between the inlet 27 and the outlet 28. Some representative components of a piston drive mechanism 20 are shown in the schematic cross sectional views depicted in FIGS. 3A-B.

The drive mechanism 20 depicted in FIGS. 3A-B, includes and inlet 27, an outlet 28, a stationary portion 320 forming a channel 35, and an actuator having a piston 44 and an armature 42. In FIG. 3A, the piston 44 is in the retraced position. In FIG. 3B, the piston 44 is in the forward position. In various embodiments, the stationary portion 320 forming the channel 35 is integrally formed with a housing of the drive mechanism. The channel 35 has a proximal end 351, nearer the inlet 27, and a distal end 352, nearer the outlet 28. The piston 44 is positioned in, and axially movable within, the channel 35 to drive infusion medium out of the distal end 352 of the channel 35. A clearance 410 is formed between the piston 44 and the channel 35. Preferably, the clearance 410 is sufficiently small such that fluidic resistance between the piston 44 and the channel 35 causes a volume of infusion medium delivered during the forward portion of the pumping stroke to be greater than a volume of the infusion medium backflowing through the channel 35 during the retracting portion of the pumping stroke. The intended fluid flow path is indicated by arrows in FIGS. 3A-B. In various embodiments, the radial clearance 410 is about 0.00025 inches (0.0064 millimeters). The armature 42 is operably coupled to the piston 44 and has first 410 and second 420 opposing major surfaces. The piston 44 extends orthogonally from the first major surface 410 of the armature 42. In various embodiments, piston 44 is integrally formed with armature 42. The armature 42 and piston 44 together form an actuator.

As the piston 44 retracts (e.g., moves from the position shown in FIG. 3B to the position shown in FIG. 3A), infusion medium is drawn through inlet 27 to the piston channel 35. As the piston 35 is advanced, infusion medium is forced out of the channel 35 and out of the outlet 28. Electromagnetic energy causes the armature 42 to advance the piston 44 in the channel 35. A coil or solenoid may be disposed in the stationary member 320 forming the channel 35 to provide such electromagnetic energy. A mechanical biasing member (not shown), such as a spring, or electromagnetic force may allow the piston 44 to retract. Absent increased fluidic resistance between the stationary member 320 and the armature 42 as the armature 42 approaches the stationary member 320, the armature 42 would increase speed as the piston 44 advances in the channel 35 due to increased electromagnetic force as the armature 42 moves closer to the stationary member 320 housing the coil. The armature 42 might then contact the stationary member 320 at a high velocity causing an audible noise. However, the increased fluidic resistance between the armature 42 and the stationary member 320 as the armature 42 approaches the stationary member 320 counteracts the acceleration of the armature 42 as the armature 42 approaches the stationary member 320, thus resulting in more quiet operation of the drive mechanism 20. If fluidic resistance is high, increased energy consumption would be expected to be needed to overcome the resistance as the piston 44 advances. As described in more detail below, the armature 42 may be configured to maintain low energy consumption while operating quietly.

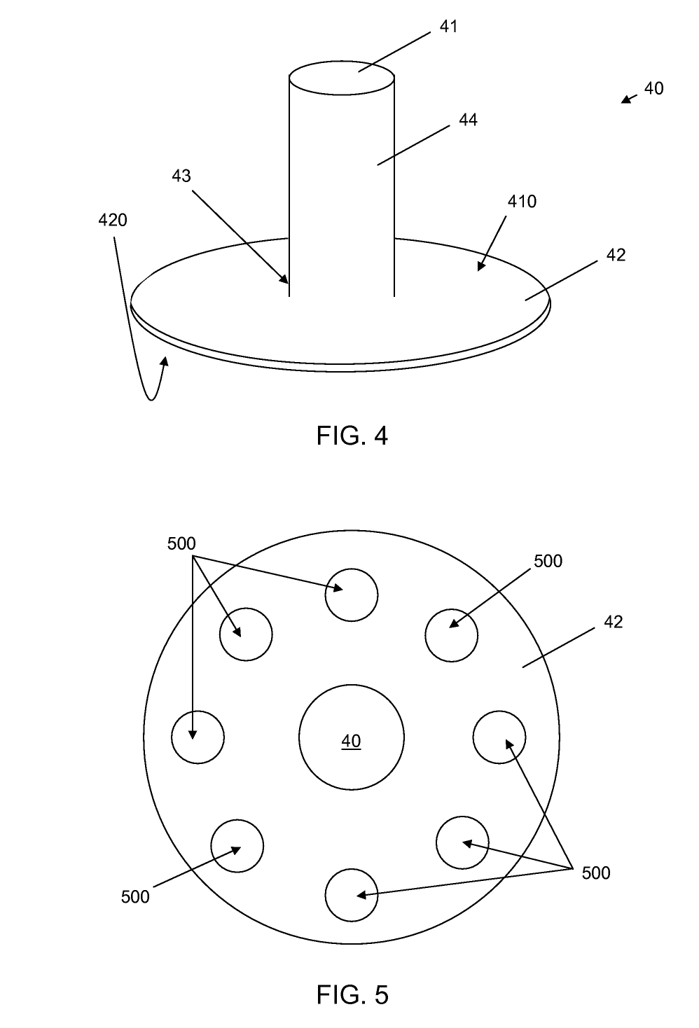

Referring now to FIGS. 4-11, representative embodiments of actuators 40 having pistons 44 and armatures 42 are shown. The pistons 44 have a proximal end 43 and a distal end 41. The armature 42 is operably coupled to the proximal end 43 of the piston 44. In various embodiments, armature 42 includes a plurality of openings 500 or vents that extend through the armature 42 from the first major surface 410 to the second major surface 420. The openings 500 or vents may be in any suitable shape or size. By way of example, the openings 500 may be generally circular, square, ellipsoidal, rectangular, triangular, tear drop shaped, or the like. Each opening 500 may be the same size or vary in size. The cumulative size of the surface area of the openings 500 may vary with the surface area of the major surfaces 410, 420 of the armature 42. As the surface area of the major surfaces 410, 420 of the armature 42 increases, the cumulative surface area of the vents 500 may also increase. In various embodiments, the surface area of the vents 500 occupy between about 20% and about 40% of the surface area of the first 410 or second 420 major surface of the armature 42. For example, if armature 42 has a round shaped first major surface having a diameter of 226 square millimeters, the cumulative area of the openings occupying the first major surface may be between about 45 square millimeters and about 90 square millimeters. In some embodiments, the surface area of the vents 500 occupy between about 25% and about 35% of the surface area of the first 410 or second 420 major surface of the armature 42, for example, between about 27.5% and about 32.5%, or about 30%. For the purposes of the present disclosure, the surface area of the first 410 or second 420 major surface of the armature 42 may be calculated by assuming the surface is flat and smooth and ignoring any surface texturing or recesses that may be present. Further, the ratio of the cumulative surface area of the openings 500 to the surface area of the first 410 or second 420 major surface of the armature 42 may be calculated by comparing the surface area of the major surface without openings 500 to the cumulative surface area of the openings 500. For example, the ratio equals the cumulative surface area of openings occupying a major surface of an actuator (assuming major surface area is smooth and flat) divided by the area of the major surface of the actuator (assuming the major surface is smooth and flat and has no openings).

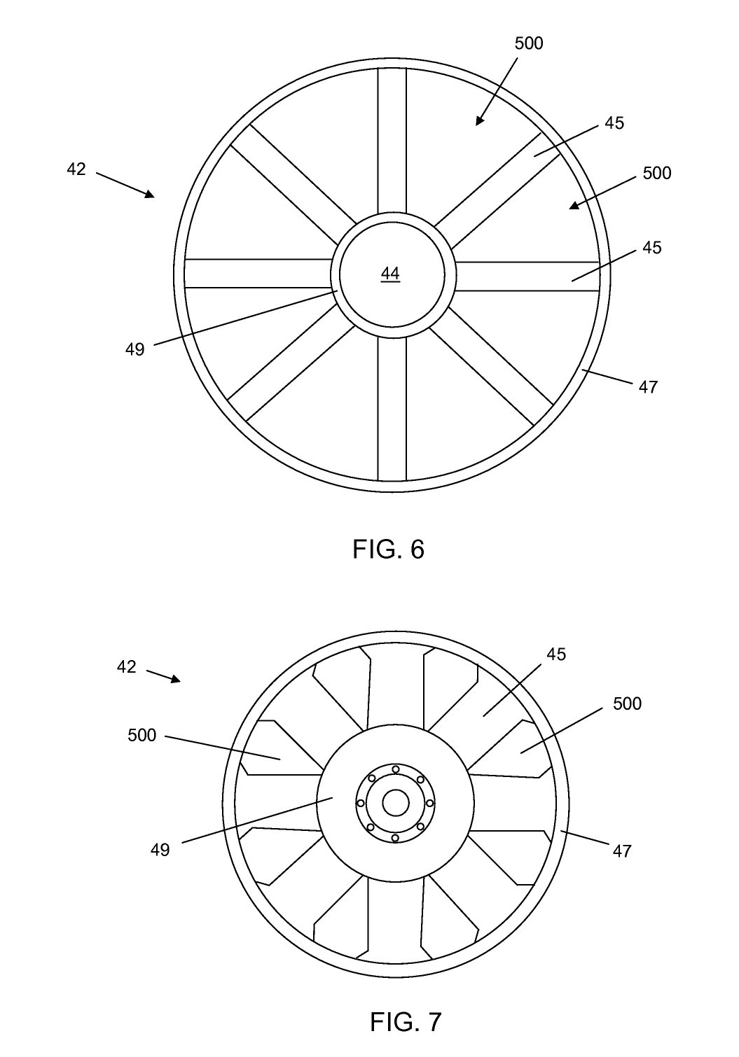

Some examples of actuators 40 having armature portions 42 with openings 500 extending through the armature 42 are shown in FIGS. 5-7 and 9-11. Referring to FIG. 5, openings 500 extending through armature 42 may be circular and equally spaced apart. Referring to FIG. 6, the armature may include a plurality of struts 45 between openings 500. The struts 45, in the depicted embodiment, extend from an inner section 49 (or inner pole) to an outer pole surface 47. Of course, openings 500 may be any other suitable shape. For example, and referring to FIG. 7, the openings 500 are shaped in a partial diamond configuration. The openings 599 in the armature 42 may, in some embodiments, be tear-drop shaped rather than having the abrupt angular changes shown in FIG. 7. In the embodiment shown in FIG. 7, the ratio of the cumulative surface area of the openings 500 to the surface area of the depicted major surface of the armature 42 is about 0.3 to 1. For example, if the diameter of the armature 42 was about 17 millimeters, the surface area of the armature would be about 226 square millimeters. If the cumulative surface area of the surface that the openings 500 occupy were about 65 millimeters, the ratio would be about 0.3 to 1. That is, the openings 500 would occupy about 30% of the total area of the major surface of the armature 42.

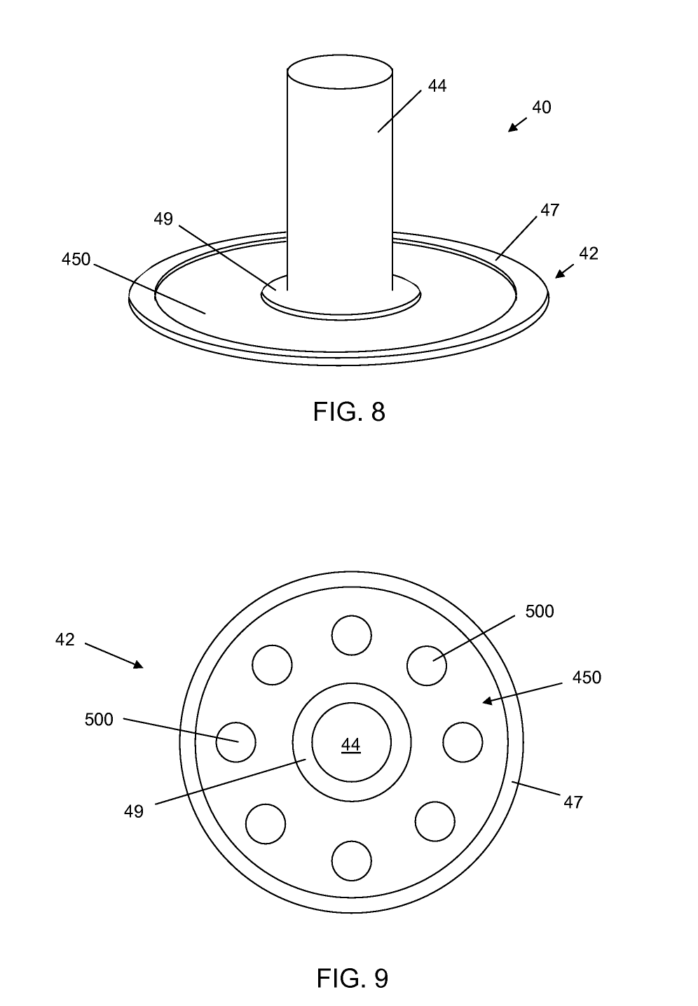

With reference to FIG. 8, the armature 42 in the depicted embodiment includes an inner section 49, an outer pole surface 47, and a recessed area between the inner pole 49 and the outer pole surface 47. In many embodiments, the surface (top surface according to orientation in FIG. 7) of the inner pole 49 and the outer pole surface 47 are coplanar. The recess 450 may be any suitable depth. In various embodiments, the depth of the recess is between about 0 millimeters and about 0.2 millimeters. With armatures 42 having a diameter of between about 15 millimeters and about 20 millimeters, the effect of the depth of the recess 450 on energy and noise were found to be negligible at depths greater than about 0.2 millimeters. The recess 450 may occupy any suitable percentage of the total surface area of a major surface of the armature 42. For example, the recessed portion 450 may occupy between about 0% and about 70% of the total area of the major surface of the armature 42, between about 35% and about 70% of the total area of the major surface of the armature 42, or between about 60% and about 70% of the total area of the major surface of the armature 42.

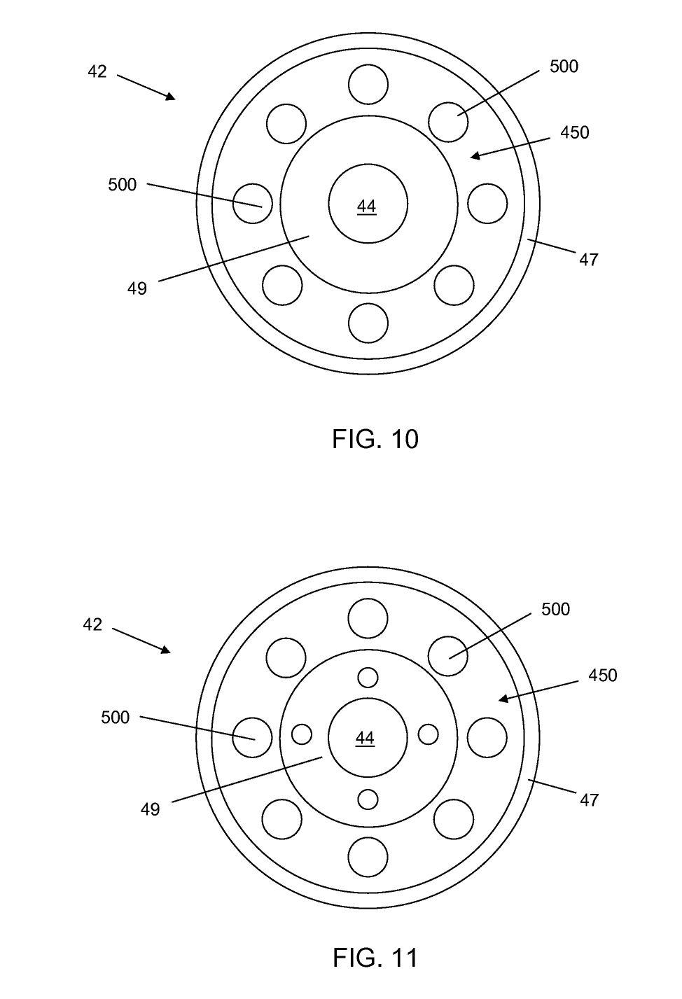

As shown in FIGS. 9-11, openings 500 may be formed in the recessed area 450. As shown in FIG, 11, openings 500′ may be formed in the inner section 49. Of course, openings may extend through the armature 42 at any suitable location. Comparing FIG. 9 with FIG. 10, the surface area of the inner pole 49 may vary to any suitable degree. By way of example, the surface area of the inner section 49 may occupy between about 85% and about 15% of the total area of the major surface of the armature 42, between about 45% and about 15% of the total area of the major surface of the armature 42, or between about 20% and about 15% of the total area of the major surface of the armature 42.

The ratio of the surface area of the inner pole 49 to surface area of the outer pole surface 49, in various embodiments, is between about 5 to 1 and about 1 to 1, between about 3 to 1 and about 1 to 1, or generally about 1 to 1.

While the armature 42 depicted in FIGS. 4-11 are generally circular and disc shaped, it will be understood that that the actuator may take any suitable shape, such as ellipsoidal, retangular, triangular, non-descriptive, or the like. A suitable shape of the armature 42 will depend in part on the shape of the coil or solenoid housed in the stationary portion of the drive mechanism.

Pistons and armatures as described herein may be made of any suitable material. For example, pistons or armatures may be formed from generally rigid, biocompatible and infusion medium compatible material, having a relatively high magnetic permeability such as, but not limited to, ferrous materials, ferritic stainless steel with high corrosion resistance, or the like. Pistons or armatures can also be fabricated with non-compatible materials and encased or plated in compatible materials. Pistons, armatures, or actuators may be molded, machined, or otherwise formed.

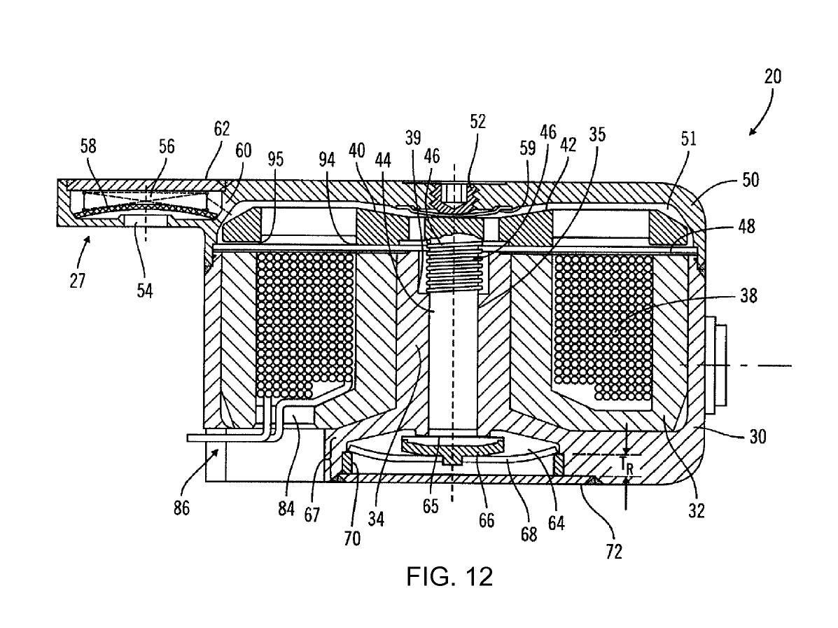

Pistons and armatures as described above, may be employed in any suitable piston drive mechanism for infusing a fluid medium. Examples of infusion devices employing piston drive mechanisms in which such actuators and armatures may be used include U.S. Patent Application Publication No. 2007/0168008, entitled “Implantable Therapeutic Substance Delivery Device Having a Piston Pump with and Anti-Cavitation Valve”; U.S. Patent Application Publication No. 2006/0206099, entitled “Low Profile Inlet Valve for a Piston Pump Therapeutic Substance Delivery Device”; U.S. Pat. No. 6,997,921, entitled “Infusion Device and Driving Mechanism for Same”, each of which is hereby incorporated by reference in their respective entireties to the extent that they do not conflict with the present disclosure. For the sake of brevity, details regarding one suitable drive mechanism and infusion device, generally as described in U.S. Pat. No. 6,997,921, is described below with reference to FIGS. 12-17.

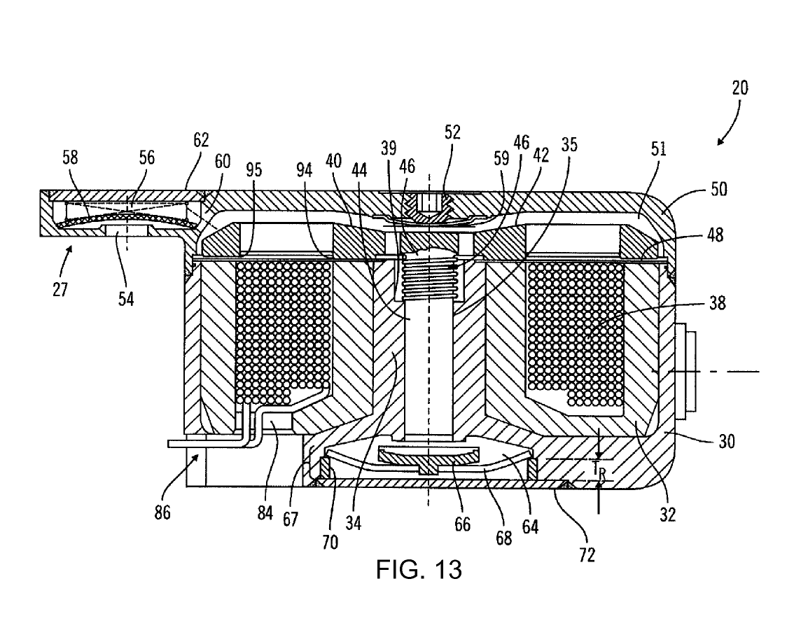

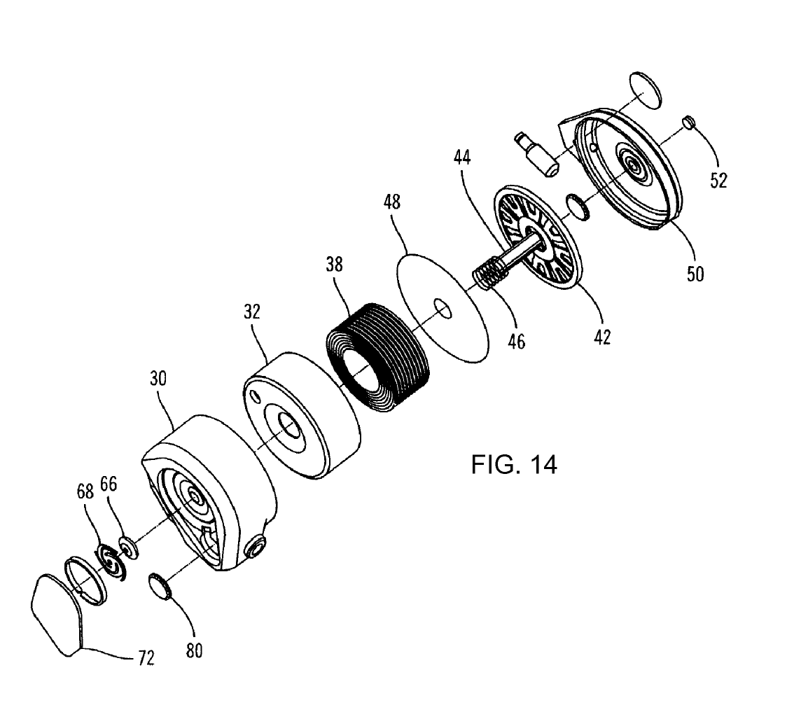

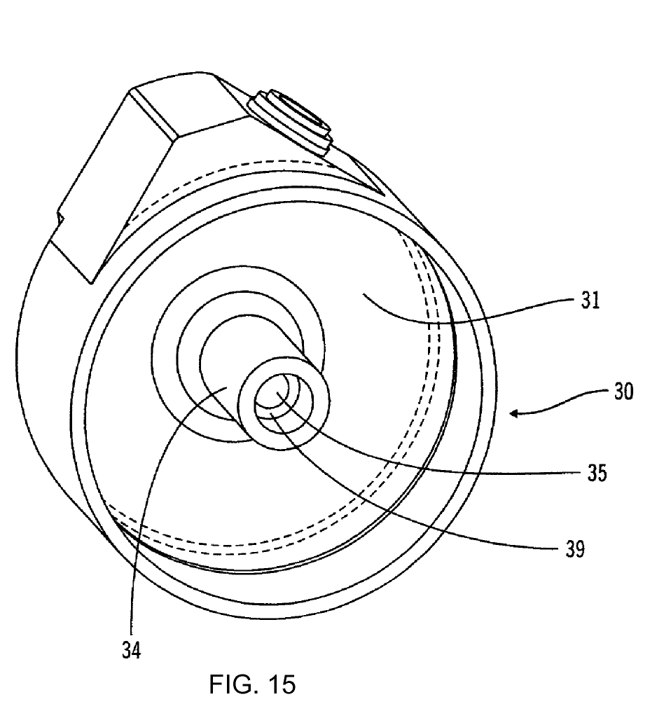

FIG. 12 shows a cross-sectional view of an embodiment of a drive mechanism 20, in a retracted position or state. FIG. 13 shows a cross-sectional view of the same drive mechanism 20 embodiment, in a forward position or state. As described in more detail below, the drive mechanism 20 employs electromagnetic and mechanical forces to change (or move) between retracted and forward states, to cause infusion medium to be drawn in through the inlet 27 and forced out of the outlet 28. The drive mechanism 20, according to one embodiment, includes an assembly of components as shown in an exploded view in FIG. 14. Some of these components are also shown in perspective views in FIGS. 15-17.

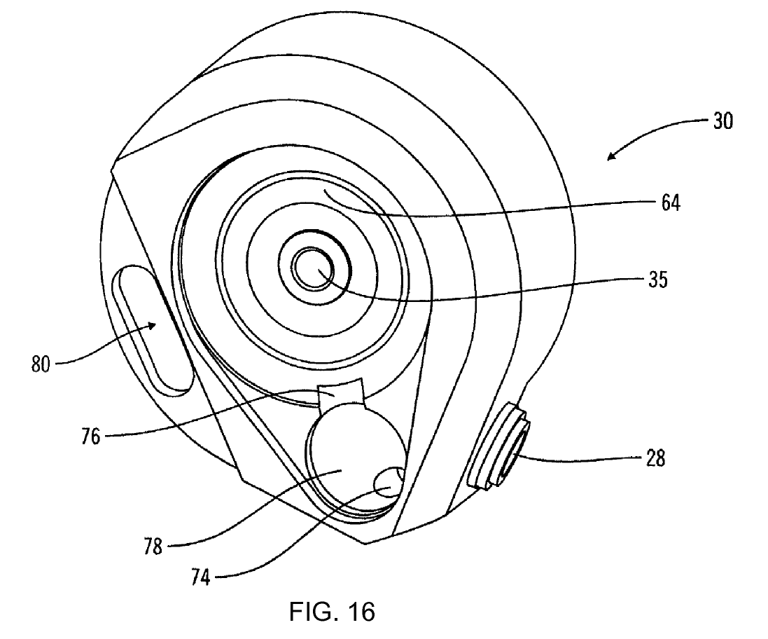

With reference to those drawings, the drive mechanism 20 includes a housing member 30 that is open on one side to a hollow, annular interior section 31. FIGS. 15-16 show two perspective views of the housing 30. The housing member 30 has a central hub portion 34 with a central piston channel 35. The bottom side of the housing member 30 (with reference to the orientation shown in FIGS. 12-13), includes an opening to the hollow interior section 31 through which coil wires may pass, as described below. The bottom side of the housing member also includes a configuration of recesses and cavities for providing an outlet chamber, an outlet passage and, in some embodiments, accumulator chambers as described below. The housing member 30 is preferably made of a generally rigid, biocompatible and infusion medium compatible material, having no or low magnetic permeability such as, but not limited to, titanium, stainless steel (which may be ferritic or non-ferritic), biocompatible plastic, ceramic, glass or the like.

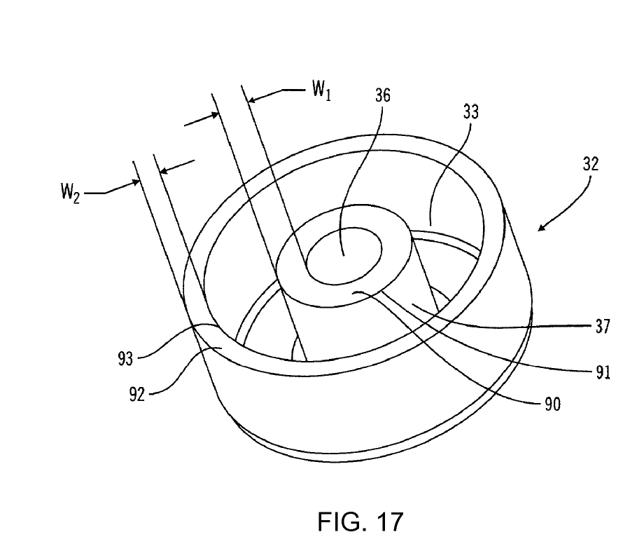

As shown in FIGS. 12-13, a coil cup 32 is located within the annular interior section of the housing 30. A perspective view of the coil cup 32 is shown in FIG. 17. The coil cup 32 has a generally cylinder shape, open on one side to a hollow, annular interior 33. The coil cup includes an open piston channel or bore 36 located in a central hub portion 37, axial relative to the annular interior. The hub portion 37 of the cup member defines an inner annular wall 90 having an end surface 91 (or inner pole surface) of width W₁. The cup member has an outer wall 92 having an end surface 93 (or outer pole surface) of a width W₂. The outer wall 92 is connected to the inner wall 90 or hub portion 37 by a backiron portion of the cup member. As described in further detail below, at the open end of the cup member, the end surfaces 90 and 92 of the inner and outer walls 91 and 93 define pole surfaces that cooperate with pole surfaces on an armature to provide a path for electromagnetic flux during a forward stroke of the drive mechanism. In various embodiments, the width W₁ of inner pole surface 91 is greater than the width W₂ of the outer pole surface 93, to provide certain desired electromagnetic characteristics.

When assembled, the coil cup is located in the hollow interior of the housing member 30, with the central portion 34 of the housing 30 extending through the piston channel 36 of the coil cup 32, as shown in FIGS. 12-13. A coil 38 is located within the hollow, annular interior of the coil cup 32, and is disposed around the axis A of the annular interior of the coil cup 32. The coil cup 32 is provided with an opening 84, through which coil leads extend, as shown in FIGS. 12-13. The coil cup 32 may be made of a generally rigid material, having a relatively high magnetic permeability such as, but not limited to, low carbon steel, iron, nickle, ferritic stainless steel, ferrite, other ferrous materials, or the like. The coil 38 comprises a conductive wire wound in a coil configuration. The coil wire may comprise any suitable conductive material such as, but not limited to, silver, copper, gold or the like, with each turn electrically insulated from adjacent turns and the housing. In various embodiments, the coil wire has a square or rectangular cross-section, to allow minimal space between windings, thereby to allow a greater number of coil turns and, thus, improved electrical efficiency.

The drive mechanism 20 also includes an actuator member 40, which has an armature portion 42 and a piston portion 44. The actuator member may be made of a generally rigid, biocompatible and infusion medium compatible material, having a relatively high magnetic permeability such as, but not limited to, ferrous materials, ferritic stainless steel with high corrosion resistance, or the like. In the embodiment of FIGS. 12-13, the actuator (with an armature portion 42 and a piston portion 44) is formed as a single, unitary structure. In some embodiments, the piston portion may be a separate structure with respect to the armature portion.

As described in more detail below, the armature 42 cooperates with the inner and outer walls of the coil cup 32, to provide a flux path for electromagnetic flux. The spacing between the pole surfaces on the armature 42 and the pole surfaces on the coil cup walls define gaps in the flux path. In some embodiments, the spacing between the outer pole surface 47 of the armature 42 and the outer pole surface 93 of the outer wall 92 of the coil cup 32 (or the barrier 48) is greater than the spacing between the inner pole surface 49 of the armature and the pole surface 91 of the inner wall 90 of the coil cup (or the barrier 48), when the actuator is in the retracted position shown in FIG. 12. Greater outer pole spacing, relative to the inner pole spacing, can result in reduced residual flux that could otherwise cause the armature to stick in the forward position (the FIG. 13 position). In addition, greater outer pole spacing reduces the squeezing effect on infusion medium between the outer pole of the armature 42 and the barrier 48, as the armature 42 moves toward the forward position during actuation of the pump mechanism.

Radial struts 45 in the armature 42 (see e.g. FIGS. 6-7) provide radial paths for electromagnetic flux between the outer and inner pole sections 47 and 49 of the armature 42. The openings 41 and 43 provide a passage for infusion medium to pass, as the actuator 40 is moved between retracted and forward stroke positions, to reduce resistance to the actuator motion that the infusion medium may otherwise produce. To reduce viscous resistance during actuator motion in the forward stroke direction, the inner or outer pole sections 47, 49 may have textured surfaces facing the coil cup 38, to provide flow areas for medium between the pole sections 47, 49 and the coil cup 38 (or barrier 48 described below).

With reference to FIGS. 12-13, the actuator member 40 is arranged with the piston portion 44 extending through the axial channel 35 of the housing 30 and with the armature portion 42 positioned adjacent the open side of the coil cup 32. An actuator spring 46 is positioned to force the armature portion 42 of the actuator 40 in the direction away from the open side of the coil cup 32, to provide a gap between the armature 42 and the open side of the coil cup 32. A biocompatible and infusion medium compatible barrier 48 is located over the open side of the coil cup 32, between the armature 42 and the coil cup 32, to maintain a gap between those two members and/or to help seal the annular interior of the coil cup and coil 38. In embodiments in which infusion medium may contact the coil, the barrier 48 may be omitted.

The actuator spring 46 in the illustrated embodiment comprises a coil spring disposed around the piston portion 44 of the actuator 40, adjacent the armature portion 42. One end of the coil spring abuts the armature portion 42 of the actuator, while the opposite end of the coil spring abuts a shoulder 39 in the piston channel 35 of the housing 30. In this manner, the actuator spring 46 imparts a spring force between the housing and the actuator 40, to urge the actuator toward its retracted position shown in FIG. 12.

In the illustrated embodiment, by using a coil spring 46 located around and coaxial with the piston portion 44 and disposed partially within the piston channel 35, the actuator spring may have minimal or no contribution to the overall thickness dimension of the drive mechanism. However, in other embodiments, actuator springs may have other suitable forms and may be located in other positions suitable for urging the actuator toward its retracted position shown in FIG. 12. The actuator spring 46 is preferably made of a biocompatible and infusion medium compatible material that exhibits a suitable spring force such as, but not limited to, titanium, stainless steel, MP35N cobalt steel or the like.

The drive mechanism 20 further includes a cover member 50 which attaches to the housing member 30, over the open side of the housing member and the barrier 48. The cover member 50 is preferably made of a generally rigid, biocompatible and infusion medium compatible material, having a relatively low magnetic permeability (being relatively magnetically opaque) such as, but not limited to, titanium, stainless steel, biocompatible plastic, ceramic, glass or the like.

The cover member 50 defines an interior volume 51 between the barrier 48 and the inner surface of the cover member. The armature portion 42 of the actuator member 40 resides within the interior volume 51 when the cover is attached to the housing, as shown in FIGS. 12-13. As described below, the armature 42 is moveable in the axial direction A within the volume 51, between a retracted position shown in FIG. 12 and a forward stroke position shown in FIG. 13. This movement is created by the action of electromagnetic force generated when a current is passed through the coil 38 and the mechanical return action of the actuator spring 46.

An adjusting plunger 52 is located within the cover 50, for contacting the armature 42 when the armature is in the fully retracted position shown in FIG. 12, to set the retracted position of the armature. In some embodiments, a seal may be disposed between the plunger 52 and the cover member 50, for example, but not limited to, a silicon rubber sealing ring. In further embodiments, a flexible diaphragm 59 (such as, but not limited to, a thin titanium sheet or foil) may be coupled to the inside surface of the cover 50 and sealed around the opening through which the plunger 52 extends. The diaphragm will flex to allow the plunger to define an adjustable retracted position and, yet, provide sealing functions for inhibiting leakage at the interface between the plunger 52 and the cover 50. In further preferred embodiments, once a proper armature position is set, the plunger is fixed in place with respect to the cover member, for example, by adhering the plunger to the cover member with one or more welds, adhesives or other securing methods. In further embodiments, the adjustment plunger is eliminated and retracted position is set with a fixed mechanical member acting as the retraction stop.

The cover member 50 includes the inlet 27 of the drive mechanism, which has an inlet opening 54 in fluid flow communication with the interior volume 51, as described below.

The inlet opening 54 connects in fluid flow communication with the reservoir of the infusion device 10 (FIG. 1), to receive infusion medium from the reservoir. Connection of the inlet opening 54 and the reservoir may be through suitable conduit (not shown), such as tubing made of suitable infusion medium compatible material, including, but not limited to titaniaum, stainless steel, biocompatible plasitc, ceramic, glass or the like.

The inlet opening 54 provides a flow path to an inlet chamber 56 formed in the cover member 50, adjacent the inlet opening. A filter or screen member, such as a porous or screen material 58, may be disposed within the inlet chamber 56. The filter or screen member 58 is provided in a flow path between the inlet opening 54 and an inlet port 60 to the volume 51. A one-way inlet valve (not shown), to allow medium to flow into but not out of the interior volume 51 through the inlet, may also be provided in the flow path between the inlet opening 54 and the inlet port 60, or within the inlet port 60. The cover member 50 may be provided with an inlet cover 62 that, when removed, allows access to the inlet chamber 56 to, for example, install, replace or service a filter 58 or inlet valve, or to service or clean the inlet 27. However, in various embodiments, an inlet valve is omitted and, instead, the drive mechanism 20 is configured as a single valve mechanism, employing a single outlet valve (for example, outlet valve assembly 67 described below) and no inlet valve.

As shown in FIGS. 12-13, the piston portion 44 of the actuator 40 extends through the axial channel 35 in the housing 30, toward an outlet chamber 64 at the end of the axial channel 35. The channel 35 has an inside diameter which is larger than the outside diameter of the piston portion 44. As a result, an annular volume is defined between the piston portion 44 and the wall of the axial channel 35, along the length of the axial channel 35. Infusion medium may flow through the annular volume, from the volume 51 within the cover 50 to a piston chamber 65 located between the free end of the piston portion 44 and a valve member 66 of a valve assembly 67. In some embodiments, the radial spacing between the piston portion 44 and the wall of the channel 35 is selected to be large enough to provide a suitable flow toward the piston chamber 65 to refill the piston chamber 65 (during a return stroke of the piston portion), but small enough to sufficiently inhibit back flow of medium from the piston chamber 65 (during a forward stroke of the piston portion).

The actual radial spacing between the piston portion 44 and the wall of the channel 35 to achieve such results depends, in part, on the overall dimensions of those components, the pressure differentials created in the mechanism and the viscosity of the infusion medium. In various embodiments, the radial spacing is selected such that the volume of medium for refilling is between about 1 and 4 orders of magnitude (e.g., about 2 orders of magnitude) greater than the volume of medium that backflows through the space. Alternatively, or in addition, the radial spacing may be defined by the ratio of the diameter D_{P} of the piston portion 44 the diameter D_{C} of the channel 35, where the ratio D_{P}/D_{C} is preferably within a range of about 0.990 to about 0.995. As a representative example, a total spacing of about 400 to 600 micro-inches (0.01 and 0.015 millimeters) and, preferably, an average radial gap of about 250 micro-inches (0.006 millimeters) annularly around the piston portion 44 may be employed.

The valve assembly 67 in the embodiment of FIGS. 12-13 includes the valve member 66, a valve spring 68 and support ring 70. The valve member 66 is located within the outlet chamber 64 and, as shown in FIG. 12, is positioned to close the opening between the axial channel 35 and the outlet chamber 64, when the actuator 40 is in the retracted position. In FIG. 13, the valve member 66 is positioned to open a flow passage between the axial channel 35 and the outlet chamber 64. The valve spring 68 is located within the outlet chamber 64, to support the valve member 66. The spring 68 imparts a spring force on the valve member 66, in the direction toward piston 44, urging the valve member 66 toward a closed position, to block the opening between the axial channel 35 and the outlet chamber 64.

The valve member 66 may be made of a generally rigid, biocompatible and infusion medium compatible material, such as, but not limited to, titanium, stainless steel, biocompatible plastic, ceramic, glass, gold, platinum or the like. A layer of silicon rubber or other suitable compliant material such as ethylene propylene diene M-class (EPDM), a perfluoroelastomer (e.g., FFKM), etc. may be attached to the rigid valve member material, on the surface facing the channel 35, to help seal the opening to the channel 35 when the valve member is in the closed position shown in FIG. 12.

The valve spring 68 may be made of a biocompatible and infusion medium compatible material that exhibits a suitable spring force such as, but not limited to, titanium, stainless steel, MP35N cobalt steel or the like. In the illustrated embodiment, the valve spring 68 has a generally flat, radial or spiral configuration. In various embodiments, the spring 68 includes radial arms that contact the interior of the outlet chamber in multiple locations around the periphery of the spring, to inhibit lateral or radial motion and improve stability of the spring. In further embodiments, a conical or belleville spring may be used. In yet further embodiments, other suitable valve spring configurations may be employed, including, but not limited to helical, conical, barrel, hourglass, constant or variable pitch springs or the like.

In the embodiment of FIGS. 12-13, the valve spring 68 is spaced from a valve cover 72 by the ring 70. The valve cover 72 is sealed to the housing 30, to enclose the outlet chamber 64. The ring 70 is disposed within the outlet chamber 64, between the spring 68 and the valve cover 72. With the valve member 66 supported between the spring 68 and the opening to the channel 35, the force imparted by the spring on the valve member is dependent, in part, on the characteristics and parameters of the spring and, in part, on the position of the spring within the outlet chamber. The ring 70 and the valve cover 72 are each preferably made of a generally rigid, biocompatible and infusion medium compatible material, such as, but not limited to, titanium, stainless steel, biocompatible plastic, ceramic, glass, gold, platinum or the like.

The thickness dimension T_{R} of the ring 70 may be matched to fit within a recess within the outlet chamber, as shown in FIGS. 12-13. Alternatively, the thickness dimension T_{R} of the ring 70 may be selected to define the position of the spring 68 within the outlet chamber, by defining the distance of the spring 68 relative to the valve cover 72 and relative to the opening between the axial channel 35 and the outlet chamber 64. A larger ring thickness T_{R} will space the spring further from the valve cover 72 and closer to the opening to the axial channel 35, while a smaller ring thickness T_{R} will space the spring closer to the valve cover 72 and further from the opening to the axial channel 35. In this manner, for a given spring 68, the force imparted by the spring on the valve member 66 to close the opening to the axial channel 35 (as shown in FIG. 12) may be selected or adjusted by selecting or adjusting the ring thickness T_{R}. The ring thickness T_{R} and the spring characteristics are preferably selected to provide sufficient force to urge the valve member 66 into a suitably sealed or closed position as shown in FIG. 12, yet allow the movement force of the piston portion 44 (caused by electromagnetic force generated by the coil) to overcome the spring force and open the valve member 66 as shown in FIG. 13.

In the illustrated embodiment, the outlet chamber 64 includes a cavity in the bottom of the housing 30, as shown in FIGS. 12, 13 and 16. Thus, in the illustrated embodiment, the outlet chamber cavity is generally centered within the same housing 30 that has the cavity holding the coil cup 32 and coil 38. With such an arrangement, the configuration of the drive mechanism 20 may be made with a relatively small thickness dimension (height dimension in the orientation shown in FIGS. 12-13) without compromising structural strength, as compared to alternative configurations in which the outlet chamber is formed with a separate member coupled to the housing 30.

As shown in FIG. 16, the outlet chamber cavity 64 may be provided in flow communication with an outlet 28 through a flow passage 74 and one or more accumulator cavities 78. The flow passage 74 comprises a channel which leads to the outlet 28 of the drive mechanism 20 and, eventually, to the device outlet 16 (FIG. 1). The outlet chamber cavity 64, flow passage 76, accumulator cavities 78 and flow passage 74 provide a flow path for infusion medium to flow from the outlet chamber to the device outlet 16, under pressure induced by operation of the drive mechanism 20. As shown in FIG. 16, the accumulator cavities 78, flow passage 76 and flow passage 74 may be provided lateral to the outlet chamber cavity 64 in the housing 30 to, thus, have minimal or no additional contribution to the overall thickness dimension T of the drive mechanism than that already required by the outlet chamber cavity 64.

Each accumulator cavity 78 forms a chamber which may contain one or more flexible, sealed packets, or accumulators, containing a compressible medium. In various embodiments, each accumulator preferably comprises a packet made of a biocompatible and infusion medium compatible material of sufficient strength and flexibility to compress and expand under varying fluid pressures, such as, but not limited to stainless steel, titanium, platinum, which contains a compressible medium, such as, but not limited to a noble gas, such as argon or neon, or other suitable materials and media that provide a return pressure over a broad range of compression pressures. The accumulators may be used to help stabilize the flow rate of the drive mechanism and provide a relatively constant output pressure during drive operations, by acting as damping structures within the flow path between the outlet chamber 64 and the outlet 28. In addition, the accumulators may minimize backflow down axial channel 35 while the valve is closing or even prior to the valve closing.

A drive mechanism as shown in FIGS. 12-13 may be constructed by providing components as shown in FIG. 14 and assembling the components in any suitable sequence. The components may be made according to any suitable process including, but not limited to molding, machining, extruding, sintering, casting, combinations thereof or the like.

The coil 38 may be inserted into the annular interior 33 of the coil cup 32, with the coil leads extended through a coil lead opening 84 in the coil cup. The coil may be impregnated or partially impregnated with a fill material of epoxy or the like, for adhering the coil to the coil cup and for sealing or partially sealing the coil. The fill material may also be used to adhere the barrier plate to the coil members, to avoid warping or bulging of the barrier plate after assembly.

The coil cup 32 and coil 38 may be inserted into the interior 31 of the housing 30, with the coil leads (which may be wire leads or flexible conductive tabs) extending through a coil lead opening 86 in the housing 30. In some embodiments, the coil cup and housing are configured to provide a tight, friction fit there between, without requiring additional means of adhering the two components together. In other embodiments, the coil cup 32 and housing 30 may be coupled together by any suitable adhesive material or other adhering methods, including, but not limited to welding, brazing, of the like.

The barrier 48 may be placed over the coil, coil cup and housing sub-assembly. The barrier 48 may be adhered to the housing by one or more adhering points or continuously along the circumference of the barrier 48, with any suitable adhesive material or other adhering methods, including, but not limited to welding, brazing, soldering or the like. Alternatively, or in addition, the barrier 48 may be held in place by a shoulder portion of the cover 50, as shown in FIGS. 12-13. In addition, as noted above, the barrier 48 may be adhered to the coil 38 by fill material in the coil. In various embodiments, the barrier 48 is held in a generally flat relation relative to the coil cup and coil. To enhance this flat relation, the coil cup and housing may be assembled together and then machined to planarize the barrier contact surfaces, prior to inserting the coil in the coil cup and prior to adding fill material to the coil.

Once the barrier 48 is placed over the coil, coil cup and housing, the actuator 40 may be added to the sub-assembly. First, however, the actuator spring 46 is placed around the piston portion 44, adjacent the armature portion 42 of the actuator. Then the free end of the piston portion 44 is passed through the axial channel 35 of the housing 30, with the armature end of the actuator arranged adjacent the barrier 48.

The cover member 50 may then be disposed over the armature end of the actuator and secured to the housing 30. In some embodiments, the cover member 50 is adhered to the housing by one or more adhering points or continuously along the circumference of the cover member 50, with one or more welds or any other suitable adhering methods, including, but not limited to adhesive materials, brazing or the like. The inlet filter 58 and inlet cover 62 may be pre-assembled with the cover member 50, prior to adding the cover member to the sub-assembly. Alternatively, the filter 58 and inlet cover 62 may be added to the cover member 50 after the cover member 50 is assembled onto the housing 30. In various embodiments, the filter 58 is disposed within the inlet chamber 56 and, then, the inlet cover 62 is adhered to the cover member 50 by one or more adhering points or continuously along the circumference of the inlet cover, with one or more welds or any other suitable adhering methods, including, but not limited to adhesive materials, brazing or the like.

The valve side of the drive mechanism may be assembled before or after the above-described components are assembled. On the valve side of the drive mechanism, the valve member 66 is disposed within the outlet chamber cavity 64 of the housing 30, adjacent the opening to the axial channel 35. The valve spring 68 is then disposed within the outlet chamber cavity 64, adjacent the valve member 66. The ring 70 is then disposed in the cavity 64, adjacent the spring 68. Any suitable number of accumulators may be placed within each of the accumulator cavities 78. The valve cover 72 may then be placed over the outlet chamber cavity 64 and accumulator cavities 78. In some embodiments, the housing 30 is provided with a recess 88 around the periphery of the cavities that form the outlet chamber cavity 64, accumulator cavities 78, outlet port 74 and flow passage 76, for providing a seat for the valve cover 72. In this manner, the valve cover 72 fits within the recess 88, flush with the housing 30. Also in various embodiments, the valve cover 72 is adhered to the housing 30 by one or more adhering points or continuously along the circumference of the valve cover, with one or more welds or any other suitable adhering methods, including, but not limited to adhesive materials, brazing or the like.

The volume of the piston chamber 65, the compression of the actuator spring 46 and the position of the actuator 40 in the retracted position shown in FIG. 12 may be adjusted by the adjusting the position of the adjusting plunger 52. In various embodiments, the adjusting plunger includes a threaded cylindrical member, which engages corresponding threads in a plunger aperture in the cover member 50, to allow adjustment in a screw-threading manner. The diaphragm 59 under the plunger 52 contacts the armature portion 42 of the actuator, inside of the cover member 50. The other end of the plunger 52 may be provided with a tool-engagement depression, for allowing engagement by a tool, such as a screw-driver, Allen wrench or the like, from outside of the cover member 50. By engaging and rotating the plunger 52 with a suitable tool, the depth that the plunger extends into the cover member 50 may be adjusted, to adjust the retracted position of the armature portion 42 relative to the barrier 48 (to adjust the gaps between the pole sections 47, 49 of the armature and pole sections formed by the coil cup 32, when the actuator is in the retracted position of FIG. 12). In some embodiments, adjustments of the plunger 52 are made during manufacture. In such embodiments, the adjusted position is determined and set by welding or otherwise adhering the plunger 52 in the adjusted position during manufacture. In other embodiments, the plunger 52 is not set and welded during manufacuture, to allow adjustment of plunger 52 after manufacture.

The resulting drive mechanism 20 may, therefore, be constructed to provide a relatively thin form factor and, yet provide a reliable operation that can deliver a relatively constant flow pressure and relatively precise volumes of infusion medium. A number of features can provide, or be combined to contribute to, reductions in the thickness form factor of the drive mechanism. For example, the coaxial arrangement of components such as the piston portion 44 and the coil 38, with a flow channel formed within the piston channel 35, can be implemented with a smaller thickness form factor (in the vertical dimension of FIGS. 12-13) than alternative arrangements in which those components are arranged adjacent each other in the thickness dimension.

Furthermore, the arrangement of an inlet volume 51 on one side of the coil 38 and an outlet chamber 64 on the opposite side of the coil 38, with a flow passage through the channel 35 in the coil 38 can also contribute to a reduction in the required thickness dimension of the drive mechanism, by allowing the coil 38 and channel 35 to share a common portion of the thickness dimension. The arrangement of the armature portion 42 to move within the inlet volume 51 allows those features to share a common portion of the thickness dimension. The arrangement of the outlet chamber 64 in a central location within the same housing that has the coil cup cavity allows those features to be formed in relatively close proximity to each other in the thickness dimension. The arrangement of the outlet chamber, outlet port and accumulator cavities in the housing 30 allows those features to share a common portion of the thickness dimension of the drive mechanism. Further features, including recessed shoulders 39 for the actuator spring 46, the use of a relatively flat valve spring 68 and general attention to minimizing thickness dimensions of components, where possible, can also contribute to reductions in the overall thickness dimension of the drive mechanism.

In addition, a number of features described herein can provide, or be combined to contribute to, the efficient use of power to, prolong the operational life of the drive mechanism. For example, a reduction in leakage of electromagnetic flux during coil energization, and, thus, a more efficient use of the flux generated by the coil, may be provided by configuring the width W₁ of the pole surface on the inner wall 90 of the cup member wider than the width W₂ of the pole surface on the outer wall 92 of the cup member. Similarly, more efficient conduction of electromagnetic flux may be provided by an actuator configured with a wider inner pole surface 49 than its outer pole surface 47. Also, more efficient conduction of electromagnetic flux may be provided by an actuator configured with radial sections 45 connecting the annular inner and outer pole surfaces 49 and 47.

In operation, the drive mechanism 20 in the depicted embodiments employs electromagnetic and mechanical forces to move between retracted (FIG. 12) and forward (FIG. 13) positions, to cause infusion medium to be drawn into and driven out of the mechanism in a controlled manner. In the retracted position, the spring 46 urges the actuator 40 toward its retracted position shown in FIG. 12. When the coil 38 is energized to overcome the spring force of spring 46, the actuator 40 moves to its forward stroke position shown in FIG. 13. The movement of the actuator between retracted and forward positions creates pressure differentials within the internal chambers and volumes of the drive mechanism 20 to draw medium into the inlet 27 and drive medium out the outlet 28.

More specifically, when the coil 38 is de-activated (not energized or not energized in a manner to overcome the spring force of spring 46), the actuator 40 is held in its retracted position (FIG. 12) under the force of the spring 46. When the coil is deactivated immediately following a forward stroke, the spring 46 moves the actuator 40 to the retracted position of FIG. 12, from the forward position shown in FIG. 13. The openings 41 and 43 in the armature portion 42 of the actuator 40 provide passages for medium to pass and, thus, reduce viscous drag on the actuator. As a result, the actuator 40 may move to its retracted position (FIG. 12) relatively quickly.

As the actuator 40 retracts, the piston portion 44 of the actuator is retracted relative to the valve member 66, such that a piston chamber 65 volume is formed between the end of the piston portion 44 and the valve member 66. The formation of the piston chamber 65 volume creates a negative pressure which draws infusion medium from the volume 51 of the cover member 50, through the annular space between the piston portion 44 and the wall of the channel 35, and into the piston chamber 65. While not shown in FIG. 12, other embodiments may include one or more channels through the piston portion 44, to provide one or more additional flow paths to the piston chamber 65.

In the retracted position, a gap is formed between each of the annular pole surfaces 91 and 93 defined by the inner and outer walls 90 and 92 of the coil cup 32 and a respective annular surfaces of the inner and outer pole sections 49 and 47 of the actuator's armature portion 42. In particular, with reference to FIG. 12, a first gap 94 is formed between the annular pole surface 91 of the inner cup member wall 90 and the annular surface of the inner pole section 49. A second gap 95 is formed between the annular surface 93 of the outer cup member wall 92 and the annular surface of the outer pole section 47.

When the coil 38 is energized (or energized in a manner to overcome the spring force of spring 46), the actuator 40 is forced in the direction to close the gaps 94 and 95 and moves to its forward position (FIG. 13) under the influence of electromagnetic flux generated by the energized coil. In particular, the coil may be energized by passing an electrical current through the coil conductor to create electromagnetic flux. The electromagnetic flux defines a flux path through the coil cup walls, across the gaps 94 and 95 and through the armature portion of the actuator. The electromagnetic flux provides an attraction force between the annular surfaces 91, 93 of the coil cup 32 and the annular surfaces of the armature's pole sections 47, 49, to overcome the spring force of spring 46 and draw the armature 42 toward the coil cup.

As the armature portion 42 of the actuator is drawn toward the coil cup 32, the piston portion 44 of the actuator is moved axially through the channel 35, in the direction toward the outlet chamber 64. With the coil energized, the piston portion 44 continues to move under the action of the armature, until a mechanical stop is reached, for example, mechanical contact of the actuator 40 with the barrier 48, a portion of the housing 30 or cover member 50. In some embodiments, the motion may continue until the return force of the spring and fluid pressure overcomes the electromagnetic force provided by energizing the coil.

The movement of the piston portion 44 towards the stopping point reduces the volume of the piston chamber 65 and increases the pressure within the piston chamber until the pressure is sufficient to overcome the force of the valve spring 68. As the valve spring force is overcome by the pressure within the piston chamber, the valve member 66 is moved toward an open position, away from the opening between the piston chamber 65 outlet chamber 64. When the valve member 66 is in the open position, medium is discharged through the outlet chamber 64 and outlet 28 (FIG. 16).

When the coil is deactivated and the piston portion 44 is moved back to its retracted position, the pressure in the piston chamber 65 reduces and the valve member 66 is reseated under the action of the valve spring 68. This prevents fluid from flowing back into the drive mechanism, through the outlet. In addition, a negative pressure is created in the piston chamber 65 to draw medium into the chamber for the next forward stroke, as described above.

In this manner, energizing of the coil 38 to move the actuator 40 to its forward position (FIG. 13) causes a measured volume of medium to be discharged from the outlet. As described above, when the coil 38 is de-energized, the actuator 40 is returned to the retracted position (FIG. 12) under the force of spring 46 and an additional volume of medium is drawn into the piston chamber 65 for the next discharging operation. Accordingly, the coil 38 may be energized and de-energized by a controlled electronic pulse signal, where each pulse may actuate the drive mechanism 20 to discharge a measured volume, or bolus, of medium. In preferred embodiments, the coil 38 may be electrically coupled to an electronic control circuit (not shown) to receive an electronic pulse signal from the control circuit for example, in response to a sensor signal, timer signal or other control signal input to the control circuit.

In preferred embodiments, when the piston motion is stopped at the end of the forward stroke, the valve-facing end of the piston portion 44 is in close proximity to the valve member 66, for example, spaced from the valve member 66 by no more than about ten percent (10%) of the piston diameter. In further embodiments, the valve facing end of the piston portion 44 is in contact with the valve member 66, at the end of the forward stroke. In this manner, gas that may be present in the infusion medium is less likely to accumulate within the piston chamber 65. More specifically, in some operational contexts, infusion medium may contain gas in the form of small bubbles that may migrate into the piston chamber 65 during filling of the piston chamber. As gas is significantly more compressible than liquid, too much gas within the piston chamber may adversely affect the ability of the drive mechanism to self prime.

In yet another embodiment the piston portion 44 may contact the valve member 66 at the end of the forward stroke and push the valve member 66 open. In this embodiment, it is less likely that gas will be trapped between the piston portion 44 and the valve member 66, and more likely that the chamber will be purged of gas.

The total ullage is the sum of (1) the volume at the valve-facing end of the piston portion 44 in a forward position (FIG. 13) and (2) the volume of the annular space between the piston portion 44 and the wall of the channel 35. In preferred embodiments, to provide self-priming properties, the total of those two volumes is selected to be about 25% of the volume of the volume 65.

When the actuator is stopped, for example, by contact with the barrier 48 or other mechanical stop structure, the coil current/voltage relationship-changes. In preferred embodiments, control electronics (not shown) are connected to detect the change in coil current or voltage and deactivate the coil when the armature reaches the stop point. In this manner, the coil may be energized for only as long as the electromagnetic flux generated by the coil is providing useful work. Once the actuator motion is stopped and no further useful work is provided by the electromagnetic flux, the coil may be deactivated to reduce or minimize power consumption requirements of the drive mechanism.

In addition, such control electronics may also adapt to altitude changes and further reduce or minimize power consumption of the drive mechanism. In particular, a differential pressure exists between the inlet and the outlet ports of the drive mechanism during operation. The differential pressure resists the motion of the actuator in the forward direction and, consequently, consumes energy. However, the differential pressure tends to reduce with increasing altitude, requiring less energy to move the actuator. By deactivating the coil when the actuator stopping point is sensed, the drive mechanism can, effectively, automatically adjust to altitude changes and provide power consumption efficiency independent of altitude in which the drive mechanism is used. Conversely, the system may provide more power if there is a blocked catheter.

Further features described above may be employed for purposes of improving efficiency in power consumption, by more efficiently using the electromagnetic flux generated by the coil during energization. For example, in preferred embodiments, the width of the first gap 94 (in the dimension from the surface 91 to the surfaces of the inner pole section 49) is less than the width of the second gap 95 (in the dimension from the surface 93 to the surface of the outer pole section 47), when the actuator is in the retracted position. Greater outer pole spacing, relative to the inner pole spacing, can result in reduced residual flux that could otherwise cause the armature to stick in the forward position (the FIG. 13 position). In addition, greater outer pole spacing reduces the squeezing effect on infusion medium within the second gap, as the armature 42 moves toward the forward position during actuation of the pump mechanism.

In various preferred embodiments, the width W₁ of the pole surface on the inner wall 90 is greater than the width W₂ of the pole surface on the outer wall 92 of the coil cup. In addition, the width W₁ of the inner pole surface 49 is greater than the width W₂ of the outer pole surface 47 of the armature, to correspond to the difference between the width of the inner wall 90 and the width of the outer wall 92 of the cup member. In some embodiments, the width of the outer pole surface 47 of the armature is slightly larger than the width of the outer pole surface of the cup member wall 92 and the width of the inner pole surface 49 of the armature is slightly larger than the width of the inner pole surface of the cup member wall 90.

When the coil 38 is energized, the attraction force generated at the gap between a pair of pole surfaces is dependent upon the area of the pole surface. Forming the outer pole surfaces with a smaller width than the inner pole surfaces can compensate for the larger diameter and, thus, the larger surface area per unit of width of the outer pole surfaces relative to the inner pole surfaces. In some embodiments, the width of the pole surfaces are selected such that the attraction force at the inner pole is approximately 2.5 times the attraction force at the outer pole. This may be accomplished by configuring the width of the outer pole surface to have a surface area of approximately 2.5 times the surface area of the inner pole surface.

In the following, non-limiting example various embodiments of the devices and methods discussed above are described in the context of studies performed to test, inter alia, energy consumption and noise of various armature configurations.

### EXAMPLE

Twelve actuators having different armature configurations were constructed. Each of the disc shaped armatures had a diameter of about 17 millimeters. The armatures had recesses of depths varying from 0 to 0.2 millimeters and different shapes and sizes of openings. The actuators were assembled in a drive mechanism substantially as described above with regard to the drive mechanism discussed with regard to FIGS. 12-17. Energy consumption associated with a stroke of the actuator was measured. Noise associated with the actuator hitting the barrier was measured with a microphone. Post hoc, two factor, high/low analysis was performed. The factors selected were vent surface area and depth of recess. Four armature designs were employed for the initial analysis—the four with the highest and lowest depths having the largest and smallest vent surface area. Predicted values for energy and noise for other armatures were then generated based on the initial analysis and compared to measured values. The analytical models from the post hoc analysis allowed theoretical prediction of performance for a design combination of high vent surface area and low recess based on a linear relationship of surface area and recess to sound pressure level and energy consumption. This combination was embodied in the construction of the actuator with armature number 1.12. Results are presented in Table 1.

TABLE 1
Predicted and Observed Energy and
Sound of Various Armature Designs

| | | Recess | | | Pre- | Pre- |
|---|---|---|---|---|---|---|
| Armature | Vent surface | Depth | Energy | SPL | dicted | dicted |
| No. | area (mm²) | (mm) | (mJ/μl) | (dB) | Energy | SPL |
| 1.11 | 26.1 | 0.2 | 3 | 66.5 | 3.00 | 66.5 |
| 1.10 | 26.1 | 0 | 3.48 | 46.2 | 3.48 | 46.2 |
| 1.9 | 7.3 | 0.2 | 3.4 | 62.5 | 3.40 | 62.5 |
| 1.8 | 7.3 | 0 | 4 | 40 | 4.00 | 40.0 |
| 1.12 | 65.5 | 0 | 2.9 | 41 | 2.39 | 59.2 |

The measured sound production level (SPL) of armature 1.12 was much lower than predicted (41 actual vs. 59.2 predicted roughly or roughly 44% lower than predicted on the decibel scale), while energy consumption was moderately higher than predicted (2.9 actual vs. 2.39 predicted or roughly 20% higher than predicted). This result is surprising, because it would have been expected that an increase in vent surface area in combination with the reduced recess depth would have resulted in a higher level of sound production based on the theoretical prediction. This is because the increased vent surface area should result in decreased fluidic resistance as the armature travels from retracted position and approaches the stationary portion of the drive mechanism. The decreased fluidic resistance would allow moving armature to travel with low resistance through the liquid between the moving armature surface and the stationary surface. This condition would have been expected to produce increased velocity at impact and thus increased sound production. In fact, this is what was observed when comparing the two armature designs with 26.1 square millimeters of vent surface area (Nos. 1.11 and 1.10) to the two armature designs with 7.3 square millimeters of vent surface area (Nos. 1.9 and 1.8). The decrease in recess depth creates a larger effective surface of contact between the moving and stationary surfaces. The liquid must be displaced as the two surfaces are squeezed together, creating a high resistance to actuator motion for the portion of actuator travel near the contact of the two surfaces. This slows actuator travel significantly prior to impact.

It is noted that increased recess depth appears to decrease energy consumption, but increase noise (compare Nos. 1.11 to 1.10 and 1.9 to 1.8). Further, increased surface area appears to decrease energy consumption, but increase noise (compare Nos. 1.11 to 1.9 and 1.10 to 1.8) However, with armature 1.12, which was the lowest recess depth and highest vent surface area combination of all the armature designs tested, the sound production level was not the highest and the energy was not the lowest. Rather the sound production level was much lower than expected and the energy consumption only slightly higher than expected. This behavior can be employed to produce a design with the preferred combination of low energy consumption and low noise level.

Armature 1.12 had a configuration substantially as depicted in FIG. 7, except that the openings 500 were less angular and more tear drop shaped.

Thus, embodiments of the REDUCED-NOISE IMPLANTABLE INFUSION DEVICE are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. A drive mechanism for delivery of infusion medium in an implantable medical device, comprising:
an inlet for receiving the infusion medium;
a piston channel for communication of infusion medium received by the inlet, the piston channel having a distal end and a proximal end, the proximal end being closer to the inlet than the distal end;
a coil surrounding the piston channel;
a piston axially moveable within the piston channel to drive infusion medium out of the distal end of the piston channel, the piston having a proximal end and a distal end; and
an armature operably coupled to the proximal end of the piston, the armature having first and second opposing major surfaces and a plurality of vents extending through the armature from the first major surface to the second major surface, the plurality of vents cumulatively occupying between about 20% and about 40% of the total surface area of the first major surface,
wherein electromagnetic interaction between the armature and the coil causes the piston to move in the channel.

2. The drive mechanism of claim 1, wherein the plurality of vents cumulatively occupy between about 25% and about 35% of the total surface area of the first major surface.

3. The drive mechanism of claim 1, wherein the plurality of vents cumulatively occupy between about 27.5% and about 32.5% of the total surface area of the first major surface.

4. The drive mechanism of claim 1, wherein the plurality of vents cumulatively occupy about 30% of the total surface area of the first major surface.

5. The drive mechanism of claim 1, wherein the total surface area of the first major surface is about 230 square millimeters and the surface area cumulatively occupied by the vents is about 65 square millimeters.

6. The drive mechanism of claim 1, wherein the armature, on the first major surface includes an inner section and an outer pole surface and a recessed area between the inner section and the outer pole surface.

7. The drive mechanism of claim 6, wherein the recessed area has a depth of 0.2 millimeters or less.

8. The drive mechanism of claim 6, wherein at least one of the plurality of openings extend through the armature in the recessed area.

9. The drive mechanism of claim 6, wherein at least one of the plurality of openings extend through the armature in the inner section.

10. The drive mechanism of claim 6, wherein at least one of the plurality of openings extend through the armature in the recessed area and at least one of the plurality of openings extend through the armature in the inner section.

11. The drive mechanism of claim 1, further an inner section, an outer pole surface, and a plurality of struts extending from the inner section to the outer pole surface.

12. An implantable infusion device comprising:
a drive mechanism having
an inlet for receiving the infusion medium;
a piston channel for communication of infusion medium received by the inlet, the piston channel having a distal end and a proximal end, the proximal end being closer to the inlet than the distal end;
a coil surrounding the piston channel;
a piston axially moveable within the piston channel to drive infusion medium out of the distal end of the piston channel, the piston having a proximal end and a distal end; and
an armature operably coupled to the proximal end of the piston, the armature having first and second opposing major surfaces and a plurality of vents extending through the armature from the first major surface to the second major surface, the plurality of vents cumulatively occupying between about 20% and about 40% of the total surface area of the first major surface,
wherein electromagnetic interaction between the armature and the coil cause the piston move in the channel;
an device inlet;
a reservoir disposed in fluid communication between the device inlet and the inlet of the drive mechanism;
an outlet in fluid communication with the distal end of the piston channel of the drive mechanism;
electronics operably coupled to the coil of the drive mechanism for controlling flow rate of the infusion medium through the piston channel.

13. The infusion device of claim 12, wherein the plurality of vents cumulatively occupy between about 25% and about 35% of the total surface area of the first major surface.

14. The infusion device of claim 12, wherein the plurality of vents cumulatively occupy between about 27.5% and about 32.5% of the total surface area of the first major surface.

15. The infusion device of claim 12, wherein the plurality of vents cumulatively occupy about 30% of the total surface area of the first major surface.

16. The infusion device of claim 12, wherein the total surface area of the first major surface is about 230 square millimeters and the surface area cumulatively occupied by the vents is about 65 square millimeters.

17. The infusion device of claim 12, wherein the armature, on the first major surface includes an inner section and an outer pole surface and a recessed area between the inner section and the outer pole surface.

18. The infusion device of claim 17, wherein the recessed area has a depth of 0.2 millimeters or less.

19. The infusion device of claim 17, wherein at least one of the plurality of openings extend through the armature in the recessed area.

20. The infusion device of claim 17, wherein at least one of the plurality of openings extend through the armature in the inner section.

21. The infusion device of claim 17, wherein at least one of the plurality of openings extend through the armature in the recessed area and at least one of the plurality of openings extend through the armature in the inner section.

22. The infusion device of claim 12, further an inner section, an outer pole surface, and a plurality of struts extending from the inner section to the outer pole surface.
